(19) United States
(12) Patent Application Publication
(10) Pub. No.: US 2022/0331236 A1
(43) Pub. Date: Oct. 20, 2022

(54) SAFER AND MORE EFFECTIVE METHODS OF TRANSMUCOSAL DELIVERY FOR RAISING BLOOD PRESSURE

(71) Applicant: Belcher Pharmaceuticals, LLC, Largo, FL (US)

(72) Inventor: Darren Rubin, Largo, FL (US)

(21) Appl. No.: 17/847,606

(22) Filed: Jun. 23, 2022

Related U.S. Application Data
(63) Continuation of application No. 15/818,056, filed on Nov. 20, 2017.
Application No. 15/818,056 is a continuation-in-part of application No. 14/323,561, filed on Jul. 3, 2014, now abandoned.

(51) Int. Cl. A61K 9/00; A61J 7/00; A61K 31/137

(52) U.S. Cl. CPC A61K 9/006; A61J 7/0053; A61K 31/137

(57) ABSTRACT

The present invention provides methods of non-gastrointestinal transmucosal drug delivery that secures, sheaths, and protects vasoconstricting and or adrenergic drug from mixing with saliva and being swallowed, thereby preventing both decreased bioavailability and loss of efficacy, and ensuring restoration of proper blood perfusion. In preferred embodiments, the adrenergic drug is epinephrine and the method treats hypotension associated with anaphylaxis.

### RELATED APPLICATION

The present application is a continuation of U.S. patent application Ser. No. 15/818,056 filed, Nov. 20, 2017, entitled SAFER AND MORE EFFECTIVE METHODS OF TRANSMUCOSAL DELIVERY FOR RAISING BLOOD PRESSURE, which is a continuation-in-part of U.S. patent application Ser. No. 14/323,561, filed Jul. 3, 2014, entitled NOVEL METHODS OF TRANSMUCOSAL DELIVERY AND SAFER IMMUNOSUPPRESSION, the subject matter of both of which is incorporated herein by reference in its entirety and for all purposes.

### FIELD OF THE INVENTION

The present invention provides novel methods of non-gastrointestinal transmucosal drug delivery that secures, sheaths, and protects drug from mixing with saliva and being swallowed, thereby preventing both decreased bioavailability and loss of efficacy, and ensuring restoration of proper blood perfusion.

### BACKGROUND OF THE INVENTION

Adequate blood pressure provides the necessary perfusion of blood to tissues so that they receive oxygen and nutrients essential to their metabolic needs. A prolonged drop in blood pressure reduces blood flow and oxygen to these tissues. Eventually, this can lead to the state of circulatory shock; whole body circulatory failure by which tissues and organs lose perfusion, leading to irreparable, ischemic cellular injury, multiple organ failure, and death. Without immediate treatment, the chances of survival are greatly diminished, and cardiac arrest and respiratory arrest can ensue.

The main components of blood pressure include cardiac output, in terms of heart rate and stroke volume, which is related to heart size and strength of ventricular contraction; along with vascular resistance to flow, which is a function of peripheral vessel vasoconstriction.

Excessive peripheral vasodilation and vessel leakiness occur in conditions like systemic inflammatory response syndrome (SIRS), and in anaphylaxis and sepsis, which lead to anaphylactic shock and septic shock. These types of shock are associated with a decreased systemic vascular resistance and are distributive in nature; a subset of circulatory shock. With anaphylaxis, a severe allergic reaction, such as to a food or insect bite, triggers massive histamine release by mast cells, resulting in vasodilation. With sepsis, a blood infection, endotoxins released from blood borne pathogens, such as gram negative bacteria, lead to low perfusion by damaging endothelial cells of the vessels to release vasodilators, such as nitric oxide. Endotoxins also activate the complement pathway to release histamine from mast cells, another vasodilator. Endotoxins also activate macrophages and neutrophils of the immune system that release cytokines for a pro-inflammatory cascade that further damages endothelial cells, leading to increased vascular permeability or leakiness of the blood vessels that can decrease intravascular fluid volume. Both conditions are associated with a significant drop in blood pressure.

Hemodynamics can at least temporarily be restored by the emergency administration of Epinephrine Injection; the chemically synthesized drug product form of this endogenous catecholamine hormone, otherwise known as adrenaline, produced by the adrenal gland.

Epinephrine acts on both alpha- and beta-adrenergic receptors. The mechanism of the rise in blood pressure is from epinephrine's three-fold actions of direct myocardial stimulation that increases the strength of ventricular contraction (positive inotropic action), an increased heart rate (positive chronotropic action), and peripheral vasoconstriction. Its action on alpha-adrenergic receptors is what lessens both the vasodilation and increased vascular permeability, which occur during anaphylaxis and sepsis.

Epinephrine Injection has a rapid onset and short duration of action. In a hospital setting, Epinephrine Injection is administered intravenously, e.g., by continuous intravenous infusion for treating hypotension associated with septic shock. But staying alive and getting to an emergency room, or having emergency medical services arrive at the scene, is not always possible in a timely manner. For individuals susceptible to allergic reactions, such as certain asthmatics and those with peanut allergy, autoinjectors of Epinephrine Injection are often prescribed and carried around for intramuscular or subcutaneous injection.

Epinephrine autoinjectors are not without issue. Firstly, they are large and bulky to carry around, especially since a second unit may be needed. So some patients may not always have the autoinjector with them. Then there are issues with storing them out of the heat (e.g., a hot glove compartment may degrade the product) and replacing them often (e.g., annually) due to a short shelf-life. Because of their high cost, some families forgo this expense altogether at great risk, while some use product well beyond its expiration date. Confusion about instructions of use with proper technique can also be an issue. If a patient triggers the device prematurely, the device becomes useless, and the dose does not get administered. Even when a patient has an epinephrine autoinjector, many are reluctant to use it because of needle fear. No doubt these autoinjectors hurt badly. The long needle is under great tension by a strong spring, and pushes outward with tremendous force. If an adult administers an autoinjector to a struggling child, cases of large cuts or gashes along the leg have been reported. And with the obesity epidemic, many experts believe autoinjector needles may not be long enough to provide intramuscular injection to all patients.

There is a tremendous need for a safer alternative, as well as, a more convenient and universal method of administering epinephrine to restore hemodynamics in an emergency situation, such as for anaphylaxis and sepsis. If epinephrine was available in tablet form, many of the above issues and limitations of epinephrine autoinjectors would be eliminated. In tablet form, several doses of epinephrine can easily be carried and associated with a much longer shelf-life than autoinjectors, and fear of needles would not deter its use. The problem is that epinephrine is not conducive to oral administration. Epinephrine quickly degrades in the gastrointestinal tract and liver, and therefore, has poor oral bioavailability when ingested.

The term transmucosal drug delivery is drug delivery through a mucous membrane. Mucous membranes, or the mucosae, comprise the linings of bodily cavities and are of mostly endodermal origin and covered in epithelium, which are involved in absorption and secretion. Most absorption by the body takes place at the intestinal mucosa after oral, or enteral, administration of food and oral medications. But other mucosa are able to absorb medications, including the oral and buccal mucosa lining the mouth, the bronchial mucosa inside the lungs, and the nasal mucosa inside the nose. This invention relates to these non-gastrointestinal mucosae, and preferably relates to non-gastrointestinal mucosae residing inside the mouth, including sublingual, sublabial, buccal, palate, and gingiva sites inside a patient's mouth.

Because the swallowing of epinephrine results in its rapid metabolization in gastrointestinal mucosa and the liver, the novelty of this invention includes methods to prevent the swallowing of epinephrine when administered by transmucosal routes inside the mouth. These methods also apply to other hemodynamic agents to raise blood pressure.

So although the sublingual route represents a potential opportunity, sublingual administration of epinephrine powder or epinephrine orally disintegrating tablets or films can be swallowed accidentally, or saliva mixing with the disintegrated or dissolved product can be swallowed. This would result in loss of epinephrine due to the degradation of epinephrine associated with the oral route and first-pass metabolism by enzymes of the gastrointestinal tract and liver.

The present invention provides novel methods of non-gastrointestinal transmucosal drug delivery of epinephrine, and or other adrenergic or vasoconstricting pharmaceutical agents (e.g., vasopressin and angiotensin II), that prevent such swallowing of drug or saliva laden drug, which would otherwise reduce bioavailability and efficacy with inconsistent and suboptimal dosing. Only the present invention provides oral transmucosal delivery that ensures blood perfusion is restored in an emergency situation. The present invention guarantees that most of the epinephrine or other active pharmaceutical ingredient is transported to the circulation inside the tongue, mouth, or cheek to reach the body's systemic circulation. In this way, the present invention provides all of the advantages and none of the disadvantages inherent in prior methods and formulations. The present invention fulfills this great medical need in a safe, reliable, and pain-free method.

### BRIEF DESCRIPTION OF THE DRAWINGS

The invention is described below with reference to several figures.

FIG. 1 is an illustration of an asymmetrical pharmaceutical formulation of a bilayer tablet having a relatively flat surface area on an active pharmaceutical ingredient side.

FIG. 2 is an illustration of a cylindrical, bottom central tablet layer or drug product component containing an active pharmaceutical ingredient portion of a drug product or formulation, surrounded by a bioadhesive, saliva-blocking border or housing.

FIG. 3 is an illustration of a conical, bottom central tablet layer or drug product component containing an active pharmaceutical ingredient portion of a drug product or formulation.

FIG. 4 is an illustration of a tablet or wafer surrounded on non-bottom sides by a globular, amorphous mass of bioadhesive/physical barrier layer.

FIG. 5 is an illustration of a tablet or wafer with an overlying bioadhesive tape or film protective layer.

FIG. 6 is an illustration of a tablet or wafer having a bioadhesive tape or film protective layer on multiple sides.

### DETAILED DESCRIPTION OF THE INVENTION

The present invention provides novel methods of non-gastrointestinal transmucosal drug delivery that sheaths and protects drug from mixing with saliva and being swallowed, thereby preventing both decreased bioavailability and loss of efficacy, and ensuring restoration of proper blood perfusion with the activity of the drug. The drug is preferably epinephrine and or other adrenergic or vasoconstricting pharmaceutical agents (e.g., norepinephrine, vasopressin, and angiotensin II).

In its simplest embodiment, said pharmaceutical formulation is adhered to an oral mucosa, and the excipients therein help protect the active ingredient from interacting with saliva beyond the site of attachment. In more complex embodiments, an additional material, such as a plastic or other polymer, which forms a physical structure or barrier that covers the pharmaceutical formulation to protect the active ingredient from interacting with saliva beyond the site of attachment. This physical structure or barrier can also help adhere the pharmaceutical formulation to the oral mucosa site and help keep it dry from saliva. This physical structure or barrier is therefore preferably impermeable to saliva.

In some embodiments, this protective layer or physical structure, such as a film, membrane, or gel, dissolves to release the pharmaceutical active ingredient for delivery, and or dissolves after the active pharmaceutical ingredient has been delivered transmucosally. The timing of such dissolving of this protective layer or physical structure can be modified to achieve the desired dosing effect, and as such, the size, thickness, and composition of said protective layer or physical structure is chosen as such. In some embodiments, this protective layer or physical structure is bioabsorbable or degradable.

In yet other embodiments, this protective layer or physical structure is non-absorbable, such as a plastic polymer, and instead must be physically removed from the oral cavity following transmucosal drug delivery, or must be swallowed and excreted by the patient following transmucosal drug delivery inside the oral cavity. When a non-absorbable protective layer or physical structure is adhered to or around the target mucosa, a warm liquid, such as water or alcohol may be required for its removal, along with rubbing the protective layer or physical structure with a finger, brush, utensil, washcloth, or another object to loosen the adhesive. In other embodiments, the adhesion can be negated by a change in pH, exposure to air, or other biologically safe solvents. For some applications, including some pediatric applications, an external wire or thread is preferably attached to the physical structure so that the physical structure can easily be removed after transmucosal drug delivery in the oral cavity by pulling on this thread or wire. This thread or wire can also tether the physical structure to prevent it from being accidentally swallowed. Likewise, an external thread or wire can be attached to the physical structure for removal after non-oral cavity applications, e.g., labial or vaginal applications of transmucosal drug delivery.

In some embodiments, this protective layer or physical structure blocks at least some of the tasting of bad/bitter tasting pharmaceutical ingredients, and as such, can also prevent additional saliva secretion from taking place when an otherwise bad/bitter taste is experienced. Taste masking excipients can also be provided in some embodiments to aid compliance and further prevent saliva secretion. This protective layer or physical structure prevents dilution with saliva outside of the mucosa site of contact, and thereby, provides a more consistent and reproducible dosage, and provides tremendous advantage over prior art transmucosal delivery in the oral cavity. This invention therefore provides much more reliable dosing than regular sublingual or buccal formulation administration, with greater bioavailability, and with less first-pass metabolism and its associated first-pass metabolites. First-pass metabolites of some drugs can be very toxic. These methods that help avoid first-pass metabolism thereby reduce toxicity associated with first-pass metabolites, as less of these metabolites are produced by this method.

The pharmaceutical formulation of the invention includes at least one dosage form chosen from the class of dosage forms including, but not limited to, a liquid, liquid droplets, a gel, a paste, a film, a biofilm, a tape, a wafer, a membrane, a sheath, a disc. The pharmaceutical formulation further includes at least one pharmaceutically acceptable excipient selected from pharmaceutical excipients including, but not limited to, carriers, solvents, solubilizing excipients, taste masking excipients, sweeteners, lipids, liposomes, adherents, bioadhesives, glycoproteins, proteins, carbohydrates, starches, waxes, polymers, charged/ionic excipients, non-charged/neutral excipients, nanoexcipients/nanoparticles, microencapsulating excipients, chelating excipients, excipients that dissolve over time, biocompatible excipients, non-absorbable excipients, and bioabsorbable excipients. The at least one pharmaceutically acceptable excipient is further chosen for the desired speed of transmucosal delivery, whether desired for immediate release and delivery, or slow release and sustained transmucosal delivery. The at least one pharmaceutically acceptable excipient is likewise chosen to aid in the passive diffusion and/or active transport of the at least one pharmaceutical active ingredient.

This invention also consists of at least two dosages of a pharmaceutical formulation. These two dosages are preferably two different dosage amounts. These at least two different dosages, in some embodiments, preferably have different releasing characteristics, for example, with layers or emulsions of excipients chosen for timed release or differential release. These at least two different dosages, in other embodiments, preferably have different size/area profiles and/or different concentrations per square unit of area in contact with a mucosal surface. Furthermore, each different dosage or dosage amount can be specific to a certain mucosal site in the oral cavity so that different dosages can be administered to different mucosae in the oral cavity. This method can allow the sites in the mouth to be rotated, so that the same mucosa site is not being dosed over and over again, so as to avoid local complications, such as localized toxicity or reduced transport at that site during a treatment regimen. Because transport efficiency and the vasculature may differ between different mucosae in the oral cavity, e.g., the palate versus buccal sites, dosages can be calibrated for these various mucosae. This method of dosage and mucosa site rotation can maintain efficient drug transport over a treatment regimen, whether formulated for active transport or passive diffusion, and whether formulated for immediate release or sustained transmucosal delivery.

In some embodiments, a collective dose is formed of smaller dosage aliquots comprised of one or more dosages from one or more different applicators. Combinations of different dosage amounts and different applicators provide different options to patients to ensure that certain mucosa are not being over treated with drug so as to reduce local side effects and to provide optimum bioavailability. These different applicator combinations can be rotated to provide the same collective (total) dose, without treating the same mucosa sites consecutively.

The invention also includes placing and adhering the pharmaceutical formulation to mucosa inside the oral cavity. The invention also includes an applicator to aid in the placement of the pharmaceutical formulation inside the mouth and preferably aids in adhering the formulation to the target mucosa site. In some embodiments, the applicator is used to administer a single dosage of said pharmaceutical formulation. In other embodiments, the applicator is used to administer multiple dosages of one or more pharmaceutical formulations. In some of these embodiments, the applicator houses and dispenses multiple dosages of one or more pharmaceutical formulations of the invention with the bioadhesive.

A preferred embodiment of the invention includes a method of non-gastrointestinal transmucosal delivery of a pharmaceutical formulation of epinephrine, including an active pharmaceutical ingredient of epinephrine and at least one pharmaceutically acceptable excipient, the method avoiding at least most first-pass metabolism by the liver by preventing saliva from mixing with this active pharmaceutical ingredient of epinephrine and being ingested; the method including administration of this pharmaceutical formulation of epinephrine inside a patient's mouth for transmucosal delivery across an at least one mucous membrane inside the patient's mouth. This method further includes adhering the pharmaceutical formulation of epinephrine to the at least one mucous membrane with a bioadhesive for transmucosal delivery. In some embodiments the bioadhesive is a mucoadhesive. This method still further includes sheathing the pharmaceutical formulation of epinephrine with a barrier to protect from mixing with saliva outside of a zone/region where the pharmaceutical formulation of epinephrine is adhered to the at least one mucous membrane, so that more than half (more than 50%) of the active pharmaceutical ingredient of epinephrine from the pharmaceutical formulation of epinephrine is prevented from being swallowed. The barrier comprises a hydrophobic layer, hydrophobic surface, physical barrier, or combination thereof at least partially surrounding the pharmaceutical formulation of epinephrine. This method is further associated with at least one of greater systemic bioavailability, lower dilution, lower side effects, or a combination thereof.

In some embodiments, epinephrine is accompanied with or replaced by an at least one other member of the class of adrenergic or vasoconstricting pharmaceutical agents. Examples of other adrenergic or vasoconstricting pharmaceutical agents of this class include norepinephrine, vasopressin, and angiotensin II). In other embodiments, epinephrine is accompanied by an at least one other pharmaceutical agent in the same pharmaceutical formulation or in a separate pharmaceutical formulation. In some other embodiments, epinephrine is accompanied by an at least one other pharmaceutical agent selected from the class of antihistamines, e.g., diphenhydramine either in the same pharmaceutical formulation or in a separate pharmaceutical formulation.

Embodiments of this invention can include any number of excipient ingredients and/or percent weight/weight of these excipient ingredients. Excipient ingredients are selected from the classes of excipients including, but not limited to, antiadherents, binders, coatings, nanoparticles, chelators, buffering agents, acid reacting excipients, alkaline reacting excipients, disintegrants, fillers, diluents, colors, lubricants, glidants, preservatives, sorbents, flavors, sweeteners, carriers, solvents, surfactants, bioadhesives, mucoadhesives, and any mixtures and combinations thereof; and can include lipids, liposomes, glycoproteins, proteins, carbohydrates, saccharides, starches, waxes, and polymers.

An at least one bioadhesive excipient is an important element to this method, which includes adhering the pharmaceutical formulation of at least one active pharmaceutical ingredient, which in preferred embodiments is epinephrine, to at least one oral mucous membrane with the at least one bioadhesive for transmucosal delivery of said at least one active pharmaceutical ingredient. The active pharmaceutical ingredient (e.g., epinephrine) portion of the drug product may be secured or adhered directly or indirectly to the oral mucosal membrane. Generally, the active pharmaceutical ingredient (e.g., epinephrine) portion of the drug product is secured in contacting abutment to the oral mucosal membrane by surrounding bioadhesive of the drug product or physical structure; an indirect adhesion of the active pharmaceutical ingredient. In some instances, however, some bioadhesive may exist in the active pharmaceutical ingredient-containing formulation, allowing direct adhesion of the active portion of the drug product in contact with the oral mucosal membrane. In some instances, the bioadhesive may be at least somewhat permeable to the active pharmaceutical ingredient. So the language, “said method further including adhering said pharmaceutical formulation of active ingredient (e.g., epinephrine) to said at least one mucous membrane with a bioadhesive for said transmucosal delivery” accounts for both direct adhesion and or indirect adhesion of the pharmaceutical formulation of active ingredient and at least one mucous membrane.

This method preferably also includes sheathing the pharmaceutical formulation of the at least one active ingredient (e.g., epinephrine) with a barrier to protect from mixing with saliva outside of a zone/region where the pharmaceutical formulation of the at least one active ingredient is adhered to the at least one oral mucous membrane, to prevent the swallowing of medicated saliva. Therefore, in preferred embodiments, the barrier at least partially comprises or is structurally and or functionally associated with the at least one bioadhesive excipient.

In many embodiments, the bioadhesive excipient will be a bioadhesive polymer, which by definition is a synthetic or natural polymer which binds, sticks, or adheres to biological target tissue, namely in this case, mucosal membranes, at least temporarily. Mucosal membranes line externally exposed passageways of the body; such as the mouth, nose, respiratory tract, gastrointestinal tract, and vagina; with mucus which helps protect and keep moist these surfaces. Most embodiments of this invention are applied to the oral mucosal membranes, although, application to other mucosal membranes can be envisioned. Mucus contains water, mineral salts, and the glycoprotein known as mucin. The sublingual gland under the tongue produces mucin. The bioadhesive can stick to these moist, often mucousy, epithelial tissue surfaces. In some embodiments the bioadhesive is a mucoadhesive, in which case, interaction and or adhesion with mucus takes place.

For bioadhesion to occur, prolonged contact between the bioadhesive polymer(s) and the mucosal must be had for binding of bioadhesive polymers to the mucin/epithelial surface. Generally, the bioadhesive polymer(s) will wet and swell over the mucosal site, such as from interaction with moisture or saliva on the mucosal surface. It is believed that bioadhesive polymer chains will interdiffuse and intermingle with the mucosal membrane, and in some instances penetrating the mucus, and forming bonds with the entangled glycoprotein chains of the mucosa thereof. Stickiness may be associated with swelling. Different forces maintain the adhesion, whether cohesive, through van der Waals force, hydrogen bonding, ionic or chemical bonding, or a combination thereof.

The properties of the bioadhesion can be controlled via the selection of the bioadhesive materials, such as selecting bioadhesive polymers with the desired concentration, flexibility, hydrophilicity, swelling, and surface tension, and size/molecular weight of the bioadhesive polymer chains. The bioadhesive or bioadhesive formulation can be solid at first, like a tablet or lozenge, a powder, or be semi-solid, like a gel, membrane, or film. In some embodiments, the bioadhesive formulation can be a liquid or viscous liquid which forms a gel or solid upon interaction with saliva. The bioadhesive or bioadhesive formulation is therefore selected from the class consisting of powders, solids, tablets, lozenges, patches, semi-solids, gels, films, tapes, viscous liquids and liquids, bioadhesives that form a gel or solid, or any combination thereof. The bioadhesive is selected to have interaction at the proper pH of the oral mucosal environment. The bioadhesive is or becomes sticky after interacting with or absorbing fluid.

In preferred embodiments, an at least one bioadhesive excipient is selected from the class of natural bioadhesive polymers and or the class of synthetic bioadhesive polymers, or analogue, derivative, or combination thereof. Some bioadhesives can be thought of as biocompatible glues, gums, pastes, or gels.

Examples of members of the class of natural bioadhesive polymers include acacia gum, alginic acid, chitosan, gelatin, guar gum, pectin, sodium alginate, sodium hyaluronate, and tragacanth.

Examples of members of the class of semi-synthetic and synthetic bioadhesive polymers include carbomers and other polyacrylics, carboxymethyl cellulose, hydroxypropyl cellulose, hydroxypropyl methylcellulose, polycarbophil, polyethylene glycol, polyvinyl alcohol, polyvinylpyrrolidone, and silicone polymers.

An example of a bioadhesive paste includes gelatin, pectin, xanthan gum, polyethylene glycol, carboxymethylcellulose, mineral oil, and optionally, one or more parabens. This is different from an adhesive oral paste formulation used for many decades containing gelatin, pectin, and carboxymethylcellulose.

An example of a bioadhesive film applied as a gel includes boric acid, hydroxypropylcellulose, propylene glycol, purified water, salicylic acid, denatured alcohol, and tannic acid.

Lactose, acacia, and magnesium stearate can be used as a bioadhesive for a buccal tablet, and can be by example, in a proportion, e.g., 10 mg, 30 mg, and 10 mg; although need not be in whole number ratios.

While the bioadhesive(s) holds the at least one active ingredient or pharmaceutical formulation of active ingredient(s) in place on the oral mucosa according to this method, the active ingredient will absorbed transmucosally either by passive diffusion, facilitated passive diffusion, active transport, pinocytosis, or a combination thereof. The sheathing of the at least one active ingredient or pharmaceutical formulation of active ingredient(s) protects against the swallowing of medicated saliva and from interfering with transmucosal absorption/delivery.

The bioadhesive layer or barrier is preferably hydrophilic on the side facing the mucosal surface, to absorb moisture from the mucus layer and become sticky; while preferably being hydrophobic and moisture repellent on the opposite side, so as to prevent other saliva from entering the zone/region of the drug product and to prevent binding to mucosa surfaces on both sides. There can be some instances where the bioadhesive layer or barrier is preferably hydrophilic on both sides, including when adhering to at least two mucosal surfaces simultaneously is desired. These instances will generally include adherence to the corner of the gingiva, where it interfaces with the cheek or floor of the mouth. It can also be desired to have the bioadhesive stick to the bottom tongue surface and the floor of the mouth simultaneously. When at least two mucosal surfaces are bound simultaneously by the same drug product dosage, it can be desirable for transmucosal delivery to also occur at the at least two mucosal surfaces simultaneously, for enhanced efficiency or quickness of transmucosal delivery. Either way, adherence to at least two mucosal surfaces simultaneously can greatly increase the total adherence, total adherence surface area, ensuring that the drug product is secure and will not be swallowed during transmucosal delivery. A larger drug dose and or extended release drug dose can be had with stronger/longer bioadhesion times. This is important since treating hypotension from anaphylaxis may require multiple doses of epinephrine, and extended release may minimize the number of repeat doses.

It is desirable for the bioadhesive of the method to rapidly adhere to the mucosa, adhere adequately or strongly, and without interfering with drug release and transmucosal delivery. It is best for the bioadhesive to not be influenced by the food and drink recently taken by the patient; minimal food effects. If the patient's mouth is dry or wet, it is desirable for the bioadhesive to bind well to the mucosa in either case.

It is also desirable for the bioadhesive to work well with drug penetration enhancers, such as having a liposomal formulation (e.g., liposomal epinephrine), or have some drug penetration properties itself for transmucosal delivery, such as containing dimethyl sulfoxide (DMSO) solvent. The bioadhesive would facilitate contact of the active ingredient (e.g., epinephrine) with the underlying surface under the mucus layer. Again, the drug product may be secured and or adhered directly and or indirectly to the oral mucosal membrane by bioadhesive.

In most preferred embodiments, transmucosal delivery of the at least one active pharmaceutical ingredient, which in preferred embodiments is epinephrine, finishes well before bioadhesion stops, such as well before mucin turnover occurs. In these embodiments, the bioadhesive can be manually removed from the patient's oral mucosa after a period of time, for example after at least 1 minute, and preferably 2 minutes. In other embodiments, the bioadhesive is a natural or safe bioadhesive that can dissolved or be chewed and swallowed or spit out after the drug has been delivered.

An example of a pharmaceutical formulation of epinephrine for transmucosal delivery, including an active pharmaceutical ingredient of epinephrine and at least one pharmaceutically acceptable excipient is Epinephrine, spray dried mannitol, crospovidone, sucralose, hydroxypropyl cellulose, and sodium stearyl fumarate. In this example and in other examples, Epinephrine can be the base form of epinephrine, i.e., Epinephrine Hydrochloride, or the acid tartrate form of epinephrine, i.e., Epinephrine Tartrate or Epinephrine Bitartrate, and or other salts or forms of epinephrine. The Epinephrine content of this formulation is in an amount of 1 mg to 100 mg, but is preferably between 2.5 mg and 60 mg of Epinephrine, USP. Mannitol can range in an amount from 100 mg to 150 mg, while crospovidone and hydroxypropyl cellulose are ideally in an amount of around 10 mg each. A sweetener such as sucralose can be added, preferably in an amount of at least 0.5 mg. Sodium stearyl fumarate is in the amount of 1 to 2 mg.

Another example of a pharmaceutical formulation of epinephrine for transmucosal delivery, including an active pharmaceutical ingredient of epinephrine and at least one pharmaceutically acceptable excipient is Epinephrine, spray dried mannitol, microcrystalline cellulose, crospovidone, sucralose, and sodium stearyl fumarate. The Epinephrine content of this formulation is in an amount of 1 mg to 100 mg, but is preferably between 2.5 mg and 60 mg of Epinephrine, USP. Mannitol can range from in an amount of 60 mg to 100 mg. Microcrystalline cellulose is in an amount of around 50 mg. Crospovidone is in an amount of 5 mg to 10 mg. A sweetener such as sucralose can be added, preferably in an amount of at least 0.5 mg. Sodium stearyl fumarate is in the amount of 1 to 2 mg.

The above two examples of a pharmaceutical formulation of epinephrine for transmucosal delivery can be in various dosage formats. In preferred embodiments it is in tablet form, such as a disintegrating tablet. In other embodiments, the epinephrine formulation represents a bottom layer of a tablet.

A saliva barrier that is a bioadhesive/bioadhesive formulation or a saliva barrier with a bioadhesive/bioadhesive formulation is adjacent to or at least partially surrounding the pharmaceutical formulations of epinephrine for transmucosal delivery as described in the above two examples. The barrier preferably includes polymers. The bioadhesive preferably includes gums. The above two epinephrine examples can therefore be associated with hydroxypropylcellulose, hydroxypropyl methylcellulose, polyvinylpyrrolidine and or propylene glycol, and Acacia or Xanthan gum as barrier/bioadhesive. Other embodiments can include pregelatinised starch. The amounts of these barrier/bioadhesive materials/excipients can vary depending on the strength and longevity of the desired bioadhesive function, and are not meant to be limiting. Some embodiments can also include menthol so the user can feel the dosage on the mucosal surface working.

Other embodiments can be envisioned whereby epinephrine or a liquid formulation thereof is spray dried onto beads or another surface, such as the bottom surface of a tablet or physical barrier. In such embodiments, epinephrine or its formulation becomes a coat or coating layer to a surface or physical barrier to this invention. The inventive methods according to this invention can be carried out in further ways, and these examples are not meant to be limiting.

Of the various embodiments of the invention and ways to perform this method, one primary aspect is that the portion of the drug product or formulation that contains active pharmaceutical ingredient, which contacts the mucosal membrane of the patient, has a preferably flat surface area on one side to make a flush contact or abutment with this mucosal membrane for efficient transmucosal delivery of the active pharmaceutical ingredient, for example, delivery of epinephrine. The portion of the drug product or formulation that contains active pharmaceutical ingredient is sheathed by a protective barrier to prevent it from mixing with saliva outside of a zone/region where the epinephrine is in contact with the mucous membrane, thereby preventing such saliva mixture from being swallowed. This barrier can comprise an at least one bioadhesive and or other excipient ingredient as part of the asymmetrical formulation, or the portion of the drug product or formulation that contains active pharmaceutical ingredient as a component or sub-component to the assembled drug product. This will be made more clear by the following illustrations. The same numerals are used throughout the following drug product figures, as each figure embodies an active pharmaceutical ingredient (e.g., epinephrine) portion of the drug product or formulation (1); a sheathing bioadhesive, preferably saliva impenetrable, physical barrier layer (2); an oral mucosa contacting/facing, adhesive side/surface (3); and a non-adhering/non-mucosal side/surface (4).

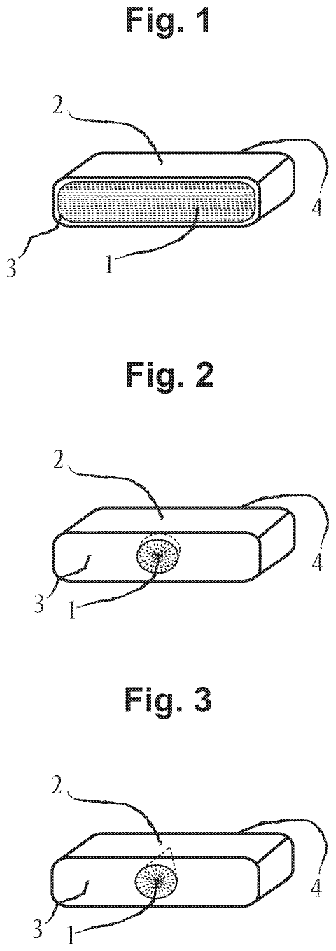

FIG. 1 is an example of an asymmetrical pharmaceutical formulation of a bilayer tablet having a relatively flat surface area on the active pharmaceutical ingredient side that will be in contact with a patient's oral mucosal membrane. This side or face of the tablet containing active pharmaceutical ingredient can be very thin, like or that of a film layer, or can have some greater thickness to it. However, the thickness of this side or face of the tablet containing active pharmaceutical ingredient is much less than the thickness of the overall tablet; i.e., preferably less than 50% the thickness of the overall tablet. The other layer or layers of the tablet contain bioadhesive excipient(s) and preferably other excipients that provide a hydrophobic layer or surface and or physical barrier to keep saliva out from mixing, and adhering the tablet to the oral mucosa, long enough time for the active pharmaceutical ingredient of the bottom layer to be taken across the oral mucosa. After transmucosal delivery of the active pharmaceutical ingredient is achieved, the remaining adhesive layer can be physically removed from the mouth, or swallowed, or dissolved then swallowed.

A second active pharmaceutical ingredient may be associated with the bottom layer or other layers. For example, norepinephrine may be contained in the bottom layer for transmucosal delivery. In another example, an antihistamine such as diphenhydramine is associated with a non-bottom layer of the tablet, which is released from this non-bottom layer and or swallowed with this non-bottom layer. Again, non-bottom layer refers to a tablet layer above the layer in contact with the oral mucosa.

In this example, saliva may exist and or be secreted from the oral mucosa area where the bottom layer is in contact with the oral mucosa. But this saliva becomes isolated or trapped between this contact layer of the tablet and the oral mucosa membrane. Saliva from surrounding areas should not interact to a considerable extent.

Other examples demonstrate a more efficient means of isolating and trapping saliva existing and or secreted from the oral mucosa area where the bottom layer is in contact with the oral mucosa, and blocking out all, nearly all, or most other saliva from surrounding areas/vicinities. In these other examples, the active pharmaceutical ingredient (e.g., epinephrine) portion of the drug product or formulation to be transmucosally delivered is exposed to the oral mucosa membrane it is (to be) in contact with, but is surrounded on all other sides by a thick border of bioadhesive(s) and or sheathing protective layer, formulation, or other drug product component. In a sense this thick border of bioadhesive and or sheathing protective layer, formulation, or other drug product component serves as a housing/impenetrable housing to hold the active pharmaceutical ingredient (e.g., epinephrine) portion of the drug product or formulation in place on the oral mucosa membrane and serves as a physical barrier to prevent mixing with saliva external to this region. FIG. 2 shows a cylindrical, bottom central tablet layer or drug product component containing the active pharmaceutical ingredient (e.g., epinephrine) portion of the drug product or formulation, surrounded by the thick bioadhesive, saliva-blocking border or housing.

During manufacture, this cylindrical, bottom central tablet layer or drug product component of active ingredient or formulation may instead be fittable and correspondingly inserted into a bottom central cavity of the drug product; or this insertion into the cavity of the border may occur during assembly when provided with a preferably, plunger-like applicator device. Plunger-like applicator devices for oral administration of a tablet have been described previously, by way of example, U.S. Pat. No. 5,292,307. However, unlike prior plunger-like applicator devices well known in the art, the present invention drug product applicator device accommodates and delivers this thick bioadhesive border with the active pharmaceutical ingredient component layer, and provides a way to hold the bioadhesive border in place at the oral mucosal membrane long enough to adhere properly. A novelty of this method is the ability of the plunger-like applicator to hold, deliver, and mount the drug product containing bioadhesive, without the bioadhesive being adhered to the applicator device itself In some embodiments, stickiness of the bioadhesive is a function of wetting, and can be useful in this applicator method.

FIG. 3 shows a conical, bottom central tablet layer or drug product component containing the active pharmaceutical ingredient (e.g., epinephrine) portion of the drug product or formulation. Different shapes of the active pharmaceutical ingredient portion of the drug product or formulation can also be envisioned.

Again, FIGS. 1-3 show a tablet-looking overall structure having either a bottom surface (i.e., FIG. 1) or a bottom central cavity (i.e., FIGS. 2 and 3) made to correspondingly hold or fit an active pharmaceutical ingredient (e.g., epinephrine) portion of the drug product or formulation as a thin or plane layer or film (i.e., FIG. 1), or as a 3-dimensional, cavity-shaped component (i.e., FIGS. 2 and 3). These figures represent a tailored fit.

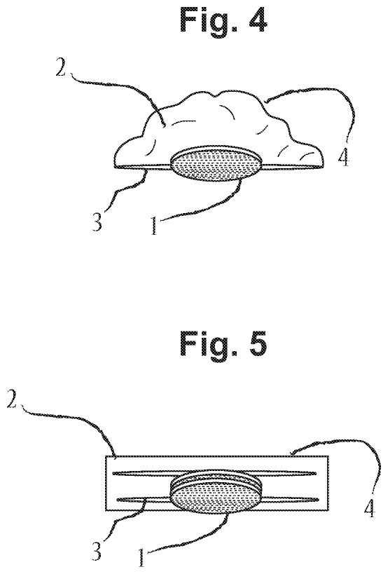

FIGS. 4 and 5 provide examples where the active pharmaceutical ingredient (e.g., epinephrine) portion of the drug product or formulation is a disintegrating and or dissolvable tablet (or wafer) that is not so custom fitted with an above sheathing bioadhesive, physical barrier layer. FIG. 4 shows the tablet (or wafer) being surrounded on non-bottom sides by a globular, amorphous mass of bioadhesive/physical barrier layer to hold the tablet in position on an oral mucosal membrane and protect against saliva mixing outside this area. FIG. 5 shows the tablet (or wafer) with an overlying bioadhesive tape or film protective layer to hold the tablet in position on an oral mucosal membrane and protect against saliva mixing outside this area. The globular bioadhesive mass or bioadhesive tape or film can be applied in the oral cavity after the tablet is placed there, but preferably, theses bioadhesive protective structures of FIGS. 4-5 are applied simultaneously with the tablet. The individual user himself or herself; or someone helping that person, such as a friend, family member, school nurse, or even passerby; can place the tablet onto the sticky bioadhesive, then use their fingers to place the bioadhesive structure with adhered tablet to an oral mucosal membrane of the mouth during a hypotensive event. Alternatively, the tablet can be pre-adhered to the bioadhesive structure and stored in a blister pack or in an applicator device.

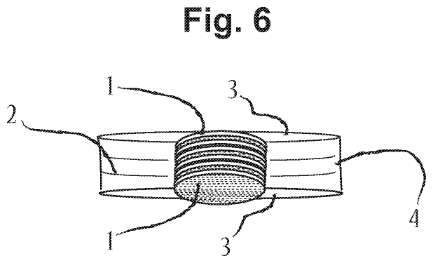

FIG. 6 shows an example of where it is desired to have the bioadhesive stick to the bottom tongue surface and the floor of the mouth simultaneously. The active pharmaceutical ingredient (e.g., epinephrine) portion of the drug product or formulation is cylindrical in shape and its top and bottom surface is exposed to both the bottom tongue surface and floor of the mouth. The bioadhesive and or barrier is preferably tube-like in shape having an inner diameter that contains the active ingredient cylinder, and a larger outer diameter to give the bioadhesive thickness, especially on its top and bottom surface to effectively stick to both mucosal surfaces. This embodiment increases total adherence (is more secure) and allows for greater and or faster transmucosal delivery by delivering to two mucosal surfaces simultaneously.

In still other embodiments, a patch (e.g., buccal patch) may be utilized, such as an epinephrine buccal patch.

The invention is a method of non-gastrointestinal transmucosal delivery of a pharmaceutical formulation of epinephrine, including an active pharmaceutical ingredient of epinephrine and at least one pharmaceutically acceptable excipient. The amount of active pharmaceutical ingredient of epinephrine (such as epinephrine base and or epinephrine bitartrate) is preferably 2.5 mg to 80 mg. Some embodiments can have a much higher epinephrine dose. For example, a slow release or controlled release embodiment can contain up to TOO mg or more of epinephrine for treating anaphylaxis so that repeat doses are not necessary. In another example, a slow release or controlled release embodiment can contain up to 1,000 mg or more of epinephrine. In this second slow release/controlled release embodiment example, plasma levels of epinephrine are maintained at a fairly constant rate over an extended period of time, to mimic intravenous infusion of epinephrine for treating hypotension associated with septic shock. There can be other disease states, such as those associated with adrenal insufficiency, that may require slow, titrated epinephrine administration.

The method avoids at least most first-pass metabolism by the liver by preventing saliva from mixing with the active pharmaceutical ingredient of epinephrine and being ingested. The method includes administration of the pharmaceutical formulation of epinephrine inside a patient's mouth for transmucosal delivery across an at least one mucous membrane inside the patient's mouth. The method further includes securing and or adhering the pharmaceutical formulation of epinephrine to the at least one mucous membrane directly and or indirectly with a bioadhesive for the transmucosal delivery. The method still further includes sheathing the pharmaceutical formulation of epinephrine with a barrier to protect from mixing with saliva outside of a zone/region where the pharmaceutical formulation of epinephrine is contacting and secured/adhered to the at least one mucous membrane so that more than half (more than 50%) of the active pharmaceutical ingredient of epinephrine from the pharmaceutical formulation of epinephrine is prevented from being swallowed. The barrier preferably comprises a hydrophobic layer, hydrophobic surface, physical barrier, or combination thereof at least partially surrounding the pharmaceutical formulation of the epinephrine. This method is further associated with at least one of greater systemic bioavailability, lower dilution, lower side effects, or a combination thereof.

The at least one mucous membrane inside the patient's mouth is chosen from at least one mucosal site inside the patient's mouth, including, but not limited to, sublingual, sublabial, buccal, palate, and gingiva sites in the patient's mouth.

With this method, less than half of the active pharmaceutical ingredient of epinephrine from the pharmaceutical formulation of epinephrine is swallowed, and preferably, less than 10 percent of the active pharmaceutical ingredient of epinephrine from the pharmaceutical formulation of epinephrine is swallowed. Even more preferably with this method, no or nearly no active pharmaceutical ingredient of epinephrine is swallowed.

The pharmaceutical formulation of epinephrine further includes at least one dosage form chosen from the class of dosage forms including powders, beads, tablets, lozenges, wafers, semi-solids, glues, gels, gums, pastes, membranes, films, tapes, liquids, or a combination thereof.

In some embodiments, the bioadhesive is a mucoadhesive.

In some embodiments, the barrier is chosen from physical structures including an absorbable/bioabsorbable layer of material that provides a physical surface to which the pharmaceutical formulation of epinephrine is attached, at least temporarily, the physical structure further protecting/sheathing the pharmaceutical formulation of epinephrine from mixing with saliva outside of a zone/region where the pharmaceutical formulation of epinephrine is contacting and or secured/adhered to the mucous membrane.

In other embodiments, the barrier is chosen from physical structures including a non-absorbable removable layer of plastic or polymer material that provides a physical surface to which the pharmaceutical formulation of epinephrine is attached, at least temporarily, the physical structure further protecting/sheathing the pharmaceutical formulation of epinephrine from mixing with saliva outside of a zone/region where the pharmaceutical formulation of epinephrine is contacting and or secured/adhered to the mucous membrane.

The method can include at least two dosages of the pharmaceutical formulation of epinephrine. The at least two dosages of the pharmaceutical formulation of epinephrine can be in two different dosage amounts/concentrations of the pharmaceutical formulation of epinephrine.

When the method includes at least two different dosages/dosage amounts/dosage concentrations of the pharmaceutical formulation of epinephrine, each of the at least two different dosages/dosage amounts/dosage concentrations of the pharmaceutical formulation of epinephrine can be specifically calibrated for transmucosal delivery to a different mucous membrane inside an oral cavity, chosen from mucosal surfaces inside a patient's mouth, including, but not limited to, sublingual, sublabial, buccal, palate, and gingiva surfaces in the patient's mouth, so that dosage administration of the pharmaceutical formulation of epinephrine can be rotated among different mucosal surfaces inside the patient's mouth to prevent local complications of drug delivery to any one such the mucosal surface during a treatment regimen, the local complications of drug delivery including localized side effects or reduced diffusion/transport.

It may be desirable for the method to be carried out with at least one dosage applicator device used to insert and place the pharmaceutical formulation of epinephrine inside of a patient's mouth.

In some embodiments, the method includes at least one dosage applicator device in the form of a bite-plate/retainer used to insert and hold at least one dosage of the pharmaceutical formulation of epinephrine inside of a patient's mouth during transmucosal delivery, and is removed after the transmucosal delivery. The pharmaceutical formulation of epinephrine is adhered to or embedded in this bite-plate/retainer.

In some embodiments, the method includes at least one dosage applicator device that dispenses an at least one dosage application of the pharmaceutical formulation of epinephrine, the method further including the use of the dosage applicator device to insert, place, and directly and or indirectly adhere the pharmaceutical formulation of epinephrine inside of a patient's mouth to a target mucous membrane therein, the applicator device dispensing the pharmaceutical formulation of epinephrine concurrently or sequentially with the bioadhesive. Storing and dispensing of bioadhesive from this applicator is unique.

The method includes at least one pharmaceutically acceptable excipient selected from classes of pharmaceutical excipients including carriers, solvents, solubilizing excipients, taste masking excipients, sweeteners, lipids, liposomes, adherents, bioadhesives, mucoadhesives, antiadherents, binders, coatings, chelators, buffering agents, acid reacting excipients, alkaline reacting excipients, disintegrants, fillers, diluents, colors, lubricants, glidants, preservatives, sorbents, flavors, carriers, surfactants, glycoproteins, proteins, saccharides, carbohydrates, starches, waxes, polymers, charged/ionic excipients, non-charged/neutral excipients, nanoexcipients/nanoparticles, microencapsulating excipients, chelating excipients, excipients that dissolve over time, biocompatible excipients, non-absorbable excipients, bioabsorbable excipients, and any combinations thereof.

In some embodiments of the method, the pharmaceutical formulation of epinephrine is adapted to be immediately released for rapid transmucosal delivery.

In other embodiments of the method, the pharmaceutical formulation of epinephrine is adapted to be released slowly for sustained transmucosal delivery.

The transmucosal delivery is by passive diffusion across a mucous membrane and or by active transport across a mucous membrane. In some embodiments, an electrical charge can be applied to the formulation via the applicator device to drive the transmucosal delivery.

In most embodiments, the method provides peripheral vasoconstriction, increased heart rate, stronger ventricular contraction, or a combination thereof in the patient.

In most embodiments, the method treats hypotension associated with anaphylaxis, anaphylactic shock, sepsis, septic shock, or a combination thereof in the patient.

In other embodiments, the method provides return of spontaneous circulation after cardiac arrest in the patient.

Interestingly, the method can provide temporary enhanced speed, physical strength, or physical endurance in the patient or user. This can have interesting applications for the military, federal agents, law enforcement agents, first responders, and even athletes. Superhuman ability may be needed in emergency situations or to accomplish above normal feats.

In some embodiments, the pharmaceutical formulation of epinephrine, including epinephrine active pharmaceutical ingredient and at least one pharmaceutically acceptable excipient, further includes at least one additional active pharmaceutical ingredient selected from vasoconstricting agents, adrenergic agents, antihistamines, or a combination thereof. In other embodiments, these other active pharmaceutical ingredients may replace epinephrine in a first or second dose.

The invention also includes a method of treating hypotension and or anaphylaxis with non-gastrointestinal transmucosal delivery of an at least one dosage of a pharmaceutical formulation of epinephrine, including preferably 2.5 mg to 80 mg of an active pharmaceutical ingredient of epinephrine, and at least one pharmaceutically acceptable excipient, the method avoiding at least most first-pass metabolism by the liver by preventing saliva from mixing with the active pharmaceutical ingredient of epinephrine and being ingested. The method includes administration of the pharmaceutical formulation of epinephrine inside a patient's mouth for transmucosal delivery across an at least one mucous membrane inside the patient's mouth. The method further includes securing/adhering the pharmaceutical formulation of epinephrine directly and or indirectly to the at least one mucous membrane with a bioadhesive for the transmucosal delivery. The method still further includes sheathing the pharmaceutical formulation of epinephrine with a barrier to protect from mixing with saliva outside of a zone/region where the pharmaceutical formulation of epinephrine is contacting and or secured/adhered to the at least one mucous membrane so that more than half (more than 50%) of the active pharmaceutical ingredient of epinephrine from the pharmaceutical formulation of epinephrine is prevented from being swallowed. The barrier preferably comprises a hydrophobic layer, hydrophobic surface, physical barrier, or combination thereof at least partially surrounding the pharmaceutical formulation of the epinephrine. The method is further associated with at least one of greater systemic bioavailability, lower dilution, lower side effects, or a combination thereof. The method includes the administration of a second dose of the pharmaceutical formulation of epinephrine after at least four minutes if hypotension and or anaphylaxis persist. In some cases, even a third dose may be required until first responders or emergency room personnel are available. In some cases, the method further includes the administration of an at least one dosage of a pharmaceutical formulation of antihistamine. The antihistamine can include diphenhydramine in some embodiments, administered by injection, orally, or oral transmucosally. In some embodiments, one of the dosages of pharmaceutical formulation of the epinephrine contains the antihistamine.

The invention also includes a method of treating hypotension and or anaphylaxis with non-gastrointestinal transmucosal delivery of an at least one dosage of a pharmaceutical formulation of epinephrine, including preferably 2.5 mg to 80 mg of an active pharmaceutical ingredient of epinephrine, and at least one pharmaceutically acceptable excipient, the method avoiding at least most first-pass metabolism by the liver by preventing saliva from mixing with the active pharmaceutical ingredient of epinephrine and being ingested. The method includes administration of the pharmaceutical formulation of epinephrine inside a patient's mouth for transmucosal delivery across at least two mucous membranes simultaneously inside the patient's mouth. The method further includes securing/adhering the pharmaceutical formulation of epinephrine directly and or indirectly to the at least two mucous membranes with a bioadhesive for the transmucosal delivery. The method still further includes sheathing the pharmaceutical formulation of epinephrine with a barrier to protect from mixing with saliva outside of a zone/region where the pharmaceutical formulation of epinephrine is contacting and or secured/adhered to the at least two mucous membranes so that more than half (more than 50%) of the active pharmaceutical ingredient of epinephrine from the pharmaceutical formulation of epinephrine is prevented from being swallowed. The barrier preferably comprises a hydrophobic layer, hydrophobic surface, physical barrier, or combination thereof at least partially surrounding the pharmaceutical formulation of the epinephrine. This method is further associated with at least one of greater systemic bioavailability, lower dilution, lower side effects, or a combination thereof.

Other variations and embodiments of the invention described herein will now be apparent to those of skill in the art without departing from the disclosure of the invention or the coverage of the claims to follow.

What is claimed is:

1. A method of treating hypotension, anaphylaxis, or both in a patient, the method comprising:
dispensing, by a plunger-like applicator device, at least two dosages of at least one pharmaceutical formulation of epinephrine, the dosages in the form of a central active pharmaceutical ingredient portion surrounded by a bioadhesive layer, and the dosages including 2.5 mg to 80 mg of epinephrine in the active pharmaceutical ingredient portion and at least one pharmaceutically acceptable excipient;
holding the at least two dosages against at least one mucous membrane using the device until the dosages are secured to the at least one mucous membrane by the bioadhesive layer; and
allowing non-gastrointestinal transmucosal delivery of the at least two dosages across at least two mucous membranes simultaneously inside said patient's mouth,
wherein said method avoids at least most first-pass metabolism by the liver by preventing more than 50% of said epinephrine from being ingested, and
wherein said method is associated with at least one of greater systemic bioavailability, lower dilution, lower side effects, or a combination thereof.

2. The method of claim 1, including sheathing said at least two dosages with a barrier to protect from mixing with saliva outside of a zone where said at least two dosages are secured to the at least one mucous membrane so that more than 50% of said epinephrine in the active pharmaceutical ingredient portion is prevented from being swallowed.

3. The method of claim 2, wherein said barrier comprises a hydrophobic layer, hydrophobic surface, physical barrier, or combination thereof at least partially surrounding said at least two dosages.
